(19) United States
(12) Patent Application Publication
(10) Pub. No.: US 2025/0248756 A1
(43) Pub. Date: Aug. 7, 2025

(54) ELECTROSURGICAL HANDPIECE AND ELECTRODE FOR AN ELECTROSURGICAL HANDPIECE

(71) Applicant: OLYMPUS WINTER & IBE GMBH, Hamburg (DE)

(72) Inventors: Christoph KNOPF, Stockelsdorf (DE); Christian BROCKMANN, Hollenstedt (DE); Jiri LAVICKA, Prerov (CZ)

(73) Assignee: OLYMPUS WINTER & IBE GMBH, Hamburg (DE)

(21) Appl. No.: 19/043,844

(22) Filed: Feb. 3, 2025

(30) Foreign Application Priority Data
Feb. 5, 2024 (DE) 10 2024 103 121.2

(51) Int. Cl. A61B 18/14; A61B 18/00

(52) U.S. Cl. CPC A61B 18/148; A61B 2018/00083; A61B 2018/0091

(57) ABSTRACT

An electrode and an electrosurgical handpiece simultaneously have a high chemical resistance and a high temperature stability. This is achieved in that an electrode for the electrosurgical handpiece consists substantially of an electrically conductive wire, with both ends of this wire being coupled to an electrode support of the handpiece. This wire is composed of at least two different kinds of material. In this case, one of these materials M₁ has a melting temperature T₁ and the second material M₂ has a melting temperature T₂. The melting temperature T₂ of the material M₂ is greater than the melting temperature T₁ of the material M₁.

The invention relates to an electrode for an electrosurgical handpiece as per the preamble of claim 1 and to an electrosurgical handpiece as claimed in claim 18.

Hand-held electrosurgical devices, in particular resectoscopes, of the type in question are used mainly in electrosurgical procedures in urology. These devices are customarily used for resection and for evaporation of tissue, for example of tissue in the lower urinary tract. To this end, the hand-held device, in particular the resectoscope, can have a longitudinally displaceable electrode support which, after the device has been inserted into the body to be treated, can be advanced with a distal working end out of a distal end of the instrument shaft of the hand-held device. An electrosurgical electrode is arranged on a distal end of the electrode support. This electrode can, for example, have the shape of a loop and, depending on the construction of the instrument, is pushed or pulled through the tissue in order to manipulate the tissue.

For the abovementioned use, a radiofrequency electric current is applied to the electrode. Known electrodes for this radiofrequency surgery are made of a homogenous metal. A distinction is made here between two groups, namely between electrodes made of metals having a high melting point, such as, for example, tungsten, and electrodes made of precious metals, such as, for example, platinum or platinum alloys. The advantage of using electrodes made of metals having a high melting temperature is that they function reliably and do not break or change their shape even at high temperatures. However, in particular electrodes made of tungsten are less reliable with respect to chemical stresses, as a result of which the life of the electrodes is reduced. In particular when using a plasma, this proves to be disadvantageous.

In contrast, precious metals do have chemical resistance but have a lower melting temperature than, for example, tungsten, this potentially proving to be disadvantageous in particular in situations in which a relatively high current density is reached. In this regard, it may happen, for example, that, during the treatment of a patient, the electrode subjected to a radiofrequency voltage comes into the vicinity of an electrically conductive implant present in the body of the patient. High electric currents occur in the event of a possible spark discharge, which can result in the electrode made of platinum melting or changing its shape in such a way that it has to be replaced. At present, there are accordingly two common variants of electrodes, which both have their advantages and disadvantages, for which reason there is no ideal electrode which does not have the mentioned disadvantages.

The object of the invention is to provide an electrode and an electrosurgical handpiece which simultaneously has a high chemical resistance and a high temperature stability.

The features of claim 1 describe how this object is achieved. Accordingly, provision is made for an electrode for an electrosurgical handpiece, possibly a resectoscope, for example, to consist substantially of an electrically conductive wire, with both ends of this wire being coupled to an electrode support of the handpiece. This wire is composed of at least two different kinds of material. In this case, one of these materials M₁ has a melting temperature T₁ and the second material M₂ has a melting temperature T₂. The melting temperature T₂ of the material M₂ is greater than the melting temperature T₁ of the material M₁. These different properties or melting temperatures of the at least two materials make it possible to rectify the aforementioned problems. With this configuration, it is possible to provide an electrode which is both chemically stable and at the same time has a high current stability, i.e. remains mechanically stable even at high current densities.

A further advantageous exemplary embodiment of the invention makes provision for the material M₁ to have a plasma resistance or a standard potential P₁ and for the material M₂ to have a plasma resistance or a standard potential P₂. In this case, the invention makes provision for P₁ to be greater than P₂. The plasma resistance or the standard potential of the materials thus now acts in a mutually opposite way, just like the melting temperature of the two materials. This choice of the materials constitutes a further possibility for providing an electrode which is chemically stable and at the same time has a high current stability.

One preferred exemplary embodiment of the invention makes provision for the material M₂ to form a core of the wire and for the material M₁ to form an outer layer encasing the core or for the material M₁ to form a core of the wire and for the material M₂ to form an outer layer encasing the core. This arrangement of the two materials makes it possible for the electrode, firstly, to be chemically stable to the outside and, secondly, to simultaneously not change its shape even at high temperatures. Different embodiments of the electrode can thus be used depending on the use or requirement.

A further conceivable exemplary embodiment of the invention can make provision for the wire to be composed of a plurality of layers of the materials M₁ and M₂ in the manner of a skin, with the materials M₁ and M₂ enveloping each other, and the core of the wire being formed of the material M₁ or M₂. This onion-like structure of the electrode makes it possible to achieve a high chemical stability while simultaneously achieving a high temperature stability of the electrode.

Moreover, the invention can make provision for the core-layer structure of the wire consisting of the at least two materials M₁ and M₂ to extend over the entire length of the wire. This shape of the electrode proves to be advantageous in particular for the production of the wire from two components since it is able to be produced inexpensively in large quantities. It is thus conceivable, for example, for one material to be enveloped by the other by a tube or to be vaporized by the corresponding material. Other production possibilities also come into consideration.

One advantageous exemplary embodiment of the invention makes provision for the wire to be composed of at least two pieces, namely a first end piece E₁ and at least one middle piece MST, with the middle piece MST being arranged against the end piece E₁, and the end piece E₁ being able to be coupled to the electrode support of the handpiece. This construction of the electrode in different portions or parts makes it possible to achieve a particularly advantageous design which provides an electrode which is chemically stable, heat resistant, and inexpensive in terms of production.

One particularly advantageous exemplary embodiment of the invention makes provision for the wire to be composed of at least three pieces, namely a first end piece E₁ and a second end piece E₂ and at least one middle piece MST, with the middle piece MST being arranged between the two end pieces E₁ and E₂, and the end pieces E₁ and E₂ being able to be coupled to the electrode support of the handpiece. This division of the electrode into different portions or parts makes it possible to achieve a particularly advantageous design which provides an electrode which is chemically stable, heat resistant, and inexpensive in terms of production.

In particular, provision is made for the middle piece MST of the wire to have a core made of the material M₂ and an encasing outer layer made of the material M₁ or for the material M₁ to form the core of the middle piece MST and for the material M₂ to form the encasing outer layer. The limitation of the materials M₁ and M₂ and in particular the limitation of this special core-layer structure on the middle piece MST makes it possible to save a considerable amount of material and thus costs. This design limits the use of the materials to the area which actually comes into contact with the body tissue. The end pieces E₁ and E₂ which do not come into contact with the tissue or with the plasma or are not used to manipulate the tissue in a targeted manner can be made of another, less expensive and more manageable material.

Preferably, it is conceivable for the middle piece MST of the wire to be composed of a plurality of layers of the materials M₁ and M₂ in the manner of a skin, with the materials M₁ and M₂ enveloping each other, and the core of the wire being formed of the material M₁ or M₂. This special exemplary embodiment makes it possible to provide an electrode which is inexpensive in terms of production, chemically stable as well as heat resistant.

The end piece E₁, or the end pieces E₁ and E₂, is/are made of an electrically conductive material M₃ and the ends E₁ and E₂ thereof connected to the middle piece MST in an electrically conductive or insulating and mechanical manner. One possibility for connecting the end pieces E₁ and/or E₂ to the middle piece MST is to glue, weld, clamp, or to crimp by means of a sleeve. This sleeve can also be welded to the two end pieces E₁ and/or E₂, with the core according to the preceding exemplary embodiments being in the interior of the sleeve. The connection possibilities mentioned here make it possible to provide an electrode which can be composed of a plurality of parts but while having the necessary chemical and mechanical stability. In this exemplary embodiment of the electrode consisting of two or three separate parts, it is conceivable for the middle piece MST to be formed of the material M₁ or M₂ and for the sleeve to be formed complementary thereto of the material M₂ or M₁. Moreover, for the exemplary embodiment of the electrode in which one of the end pieces is connected to the middle piece in an electrically insulating manner, it is conceivable for this end piece to be made of an electrically insulating material.

The wire described here can have a diameter of 0.2 mm to 1 mm, preferably of 0.28 mm. It is equally conceivable for the wire to have different dimensions. In addition, provision can be made for the outer layer encasing the core, or for the sleeve, to have a layer thickness of 3 μm to 0.2 mm, in particular 0.01 mm to 0.1 mm, preferably 0.05 mm.

It has proven particularly advantageous for the material M₁ to be platinum, or a platinum alloy, such as, for example, platinum-iridium or platinum-tungsten. The material M₂ can be tungsten, tantalum or molybdenum. Finally, it is conceivable for the material M₃ to be steel or stainless steel, copper or a copper alloy or an electrical insulator. It is expressly pointed out in this case that this list is not exhaustive; provision can rather be made for other materials having comparable properties to be used too.

An electrosurgical handpiece for achieving the mentioned object has the features of claim 18. Accordingly, provision is made for this electrosurgical handpiece, possibly a resectoscope, for example, to have an electrode as claimed in at least one of claims 1 to 17.

A preferred exemplary embodiment of the invention is described in more detail below with reference to the drawing, in which:

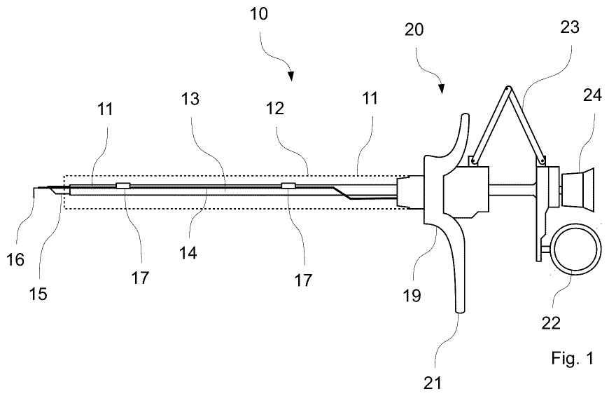

FIG. 1 shows a schematic illustration of a surgical handpiece, in particular a resectoscope,

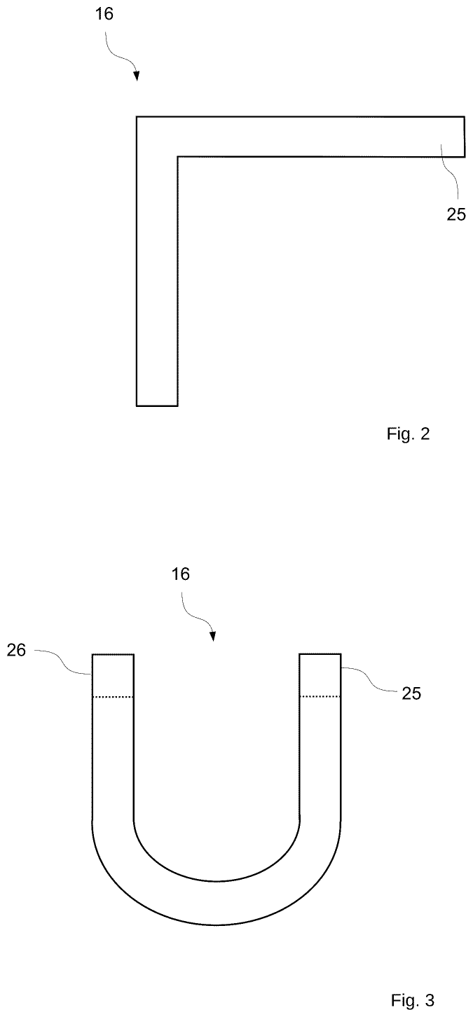

FIG. 2 shows a side view of an electrode,

FIG. 3 shows a front illustration of the electrode according to FIG. 2,

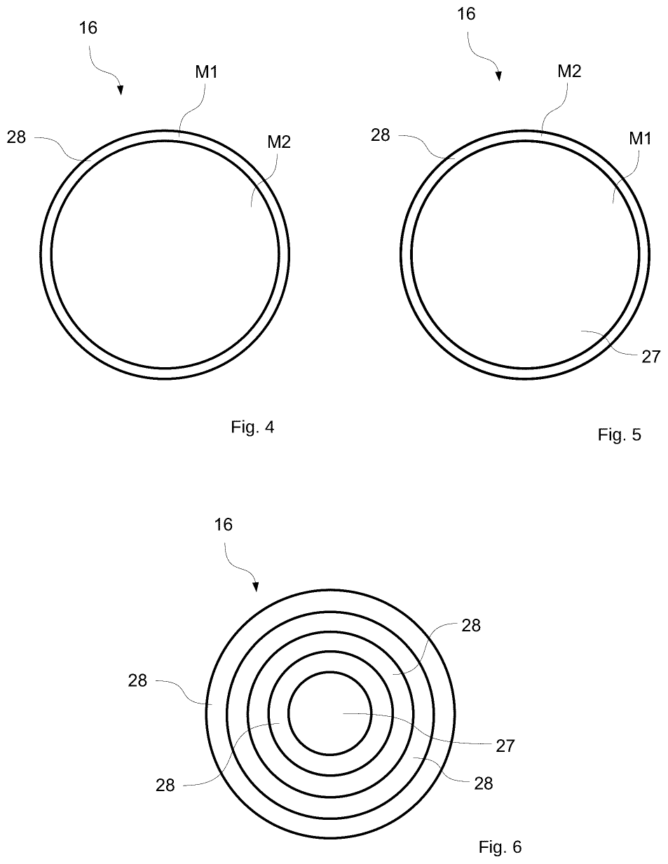

FIG. 4 shows a section through a first exemplary embodiment of the electrode,

FIG. 5 shows a section through a further exemplary embodiment of the electrode,

FIG. 6 shows a section through a further exemplary embodiment of the electrode,

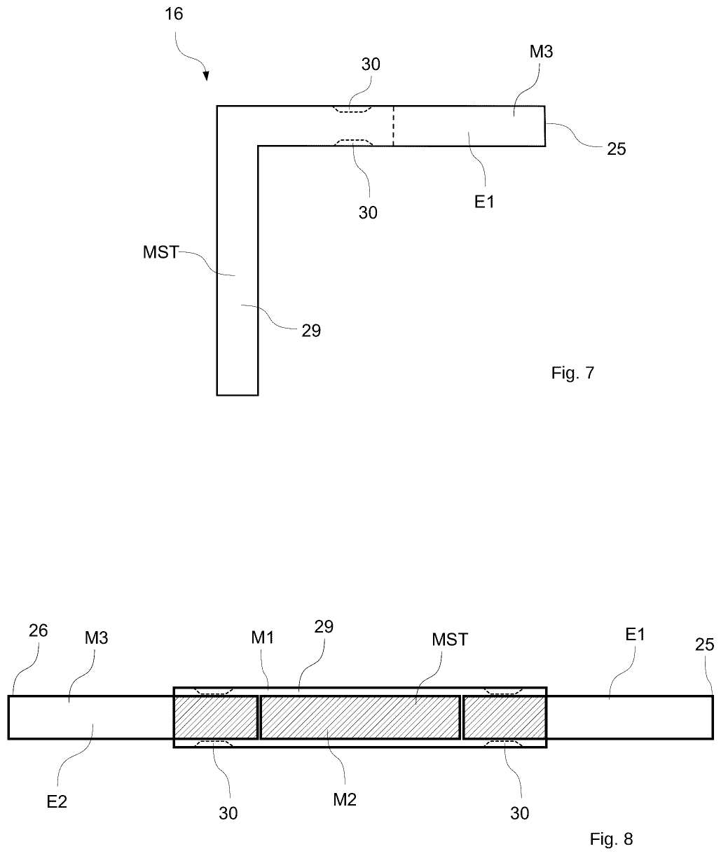

FIG. 7 shows a side view of a further exemplary embodiment of the electrode, and

FIG. 8 shows an illustration of a further exemplary embodiment of the electrode.

FIG. 1 shows a schematic cross-sectional side view of a resectoscope 10. The resectoscope 10 has a resectoscope shaft 11 which comprises an illustrated outer shaft 12 or an encasing tube. A tubular inner shaft 13 runs inside the outer shaft 12. An electrode support 14 and an indicated optical unit 15 are illustrated inside the inner shaft 13. In addition, further elements not illustrated here can be arranged in the resectoscope 10, such as, for example, a separate irrigation tube and the like.

The electrode support 14 has, at a distal end, an electrosurgical tool or an electrode 16. The electrode 16 illustrated here is in the form of a loop.

By actuation of a handle 19, the electrode support 14 can be moved in a constrained axial movement in the distal and proximal direction. It can thereby be pushed out beyond the distal end of the inner shaft 13 and of the outer shaft 12. The operator is thus also able to manipulate tissue that is located further away from the resectoscope tip. For this purpose, the inner shaft 13 and/or the electrode support 14 can also be mounted rotatably about their longitudinal axis. For the manipulation of the tissue, a radiofrequency electric current is applied to the electrode 16.

The resectoscope 10 illustrated in FIG. 1 has a passive transporter in which, by relative movement of the grip parts 21 and 22 arranged proximally on the resectoscope shaft 11, a carriage 20 is displaced in the distal direction toward the distal, first grip part 21 counter to a spring force applied by a spring bridge 23. In the displacement of the carriage 20 in the distal direction toward the grip part 21, the electrode support 14 is displaced in the distal direction in a manner not illustrated. Upon relaxation of the grip parts 21, 22, the spring force generated by the spring bridge 23 forces the carriage 20 back to its starting position, wherein the electrode support 14 is pulled in the proximal direction. Upon the return displacement of the carriage 20, an electrosurgical intervention can be performed with the electrode 16 without manual force from the operator, i.e. passively.

For the targeted treatment by means of the electrode 16, the optical unit 15 is positioned in such a way that the operator receives an optimal view of the area of the operation. For this purpose, the resectoscope 10 has, on a proximal end, an eyepiece 24 which is connected to the optical unit 15. Alternatively, it is also conceivable for a camera, instead of the eyepiece 24, to be arranged on the resectoscope 10.

The electrode 16 described here has the shape of a loop. Such a loop structure is illustrated in highly schematic fashion in FIGS. 2 and 3. In this case, a wire is shaped to form a U-shaped loop in such a way that the two ends 25 and 26 thereof are able to be electrically and optionally also mechanically coupled to the electrode support 14 of the resectoscope 10. The coupling to the electrode support 14 is generally performed by a plug connection. However, it is expressly pointed out that other electrode shapes, such as in the case of button electrodes or needle electrodes, for example, are also conceivable.

In order that the electrode 16 retains its shape or is not damaged even in the event of great heat, i.e. in the event of a large current density, which can occur when a radiofrequency AC voltage is applied to the electrode 16, it is customary to produce the wire or the electrode 16 from tungsten. As is known, a distinguishing feature of tungsten is its extremely high melting temperature. Equally, the electrode 16 needs to remain stable in shape with respect to chemical reactions which can occur in particular when using plasma, in order to thus be able to be used over the longest possible period of time without limiting the performance. This can be achieved by using platinum or a platinum alloy as the material for the wire. However, platinum is, firstly, not particularly heat resistant and, secondly, very expensive.

The electrode 16 described here consists at least of two different materials. FIG. 4 schematically shows a cross section of a possible exemplary embodiment of the electrode 16. Accordingly, provision is made for a core 27 of the electrode 16 to be formed by a material M₂ and for an outer layer 28 encasing the core 27 to be formed by a material M₁. In this case, the material M₂ has the melting temperature T₂ and the material M₁ has the melting temperature T₁. One particularly preferred exemplary embodiment makes provision for the temperature T₂ to be greater than the temperature T₁. The material M₂ could accordingly be, for example, tungsten, tantalum or molybdenum which have a high melting temperature. In contrast thereto, the material M₁ could be metals having a lower melting temperature, such as, for example, platinum, a platinum alloy, such as, for example, platinum-iridium or platinum-tungsten. By virtue of this configuration, the electrode 16 would have a high chemical resistance with respect to external influences and would simultaneously be heat resistant with respect to high current densities. Alternatively, for corresponding uses, it is also conceivable for the mentioned materials M₂ and M₁ to be arranged exactly the other way round. This is illustrated by way of example by the exemplary embodiment of FIG. 5. Therein, the outer layer 28 is formed by the material M₂ and the core 27 is formed by the material M₁.

A further possible exemplary embodiment of the invention is illustrated by FIG. 6. Here, a plurality of successive layers 28 envelope each other in the manner of an onion skin starting from a core 27. The materials M₁ and M₂ can be arranged almost arbitrarily here depending on use or requirement. Only for the sake of completeness, it is mentioned that it is also conceivable for more than two different materials to be able to be used for this core-layer structure.

In the aforementioned exemplary embodiments, the core-later structure extends over the entire length of the wire, i.e. from the end 25 to the end 26. This wire is usually 20 mm to 80 mm long. The diameter of the wire can be 0.2 mm to 1 mm, preferably 0.28 mm, while the encasing outer layer 28 can have a layer thickness of 3 μm to 0.2 mm, in particular 0.01 mm to 0.1 mm, preferably 0.05 mm.

A further exemplary embodiment of the electrode 16 is illustrated in FIGS. 7 and 8. The electrode shown therein is composed of three portions or pieces, namely a first end piece E₁, a second end piece E₂ and a middle piece MST. The middle piece MST is arranged between the two end pieces E₁ and E₂. The two end pieces E₁ and E₂ have the two ends 25 and 26 with which they are able to be coupled to the electrode support 14 in the manner described above.

One exemplary embodiment can make provision for the middle piece MST of the electrode 16 to have the same core-layer structure as described previously for the other exemplary embodiments. A further exemplary embodiment makes provision for the middle piece MST to be formed of the material M₁ or M₂. The end pieces E₁ and E₂ are in turn made of steel or stainless steel such that an electrical conductor made of tungsten, tantalum or molybdenum or of platinum, a platinum alloy, such as, for example, platinum-iridium or platinum-tungsten is situated between the two end pieces E₁ and E₂. In order to attach the middle piece MST between the two end pieces E₁ and E₂, it is conceivable for the individual pieces to be glued, welded, crimped or clamped to one another. In the exemplary embodiment illustrated in FIG. 8, a sleeve 29 is arranged at least in sections around the middle piece MST and the two end pieces E₁ and E₂. If the middle piece MST is formed of the material M₁, the sleeve 29 consists of the material M₂. Conversely, if the sleeve 29 is formed of the material M₁, the middle piece MST consists of the material M₂. In order to fasten the sleeve 29 to the end pieces E₁ and E₂, it is conceivable for the sleeve 29 to be welded or crimped to the end pieces E₁ and E₂. For this purpose, the end pieces of the sleeve 29 are provided with indentations 30 in order to connect the mentioned components of the electrode 16 to one another in a mechanically stable and electrically conductive manner. It is also conceivable for the parts to be welded and additionally crimped to one another in order to have enough stability even with respect to stronger mechanical stresses. It is expressly pointed out that the dimensioning and the arrangement, in particular of the sleeve 29, can differ from that which is illustrated only by way of example here. Apart from that, the dimensions of the electrode 16 correspond to the aforementioned values.

The last-mentioned exemplary embodiment combines the advantages of inexpensive production, extremely high heat stability and chemical resistance with respect to the external influences which act on the electrode 16 during the treatment.

List of reference signs:

| | | | |
|---|---|---|---|
| 10 | resectoscope | M₁ | material |
| 11 | resectoscope shaft | M₂ | material |
| 12 | outer shaft | E₁ | end piece |
| 13 | inner shaft | E₂ | end piece |
| 14 | electrode support | MST | middle piece |
| 15 | optical unit | | |
| 16 | electrode | | |
| 17 | guide element | | |
| 18 | longitudinal axis | | |
| 19 | handle | | |
| 20 | carriage | | |
| 21 | grip part | | |
| 22 | grip part | | |
| 23 | spring bridge | | |
| 24 | eyepiece | | |
| 25 | end | | |
| 26 | end | | |
| 27 | core | | |
| 28 | layer | | |
| 29 | sleeve | | |
| 30 | indentation | | |

1. An electrode for an electrosurgical handpiece, consisting of an electrically conductive wire, with the two ends of the wire being able to be coupled to an electrode support of the handpiece, wherein the electrically conductive wire is composed of at least two different kinds of material, namely at least one material M₁ having a melting temperature T₁ and at least one material M₂ having a melting temperature T₂, with T₂ being greater than T₁.

2. The electrode for an electrosurgical handpiece as claimed in claim 1, wherein the material M₁ has a plasma resistance or a standard potential P₁ and the material M₂ has a plasma resistance or a standard potential P₂, with P₁ being greater than P₂.

3. The electrode for an electrosurgical handpiece as claimed in claim 1, wherein the material M₂ forms a core of the wire and the material M₁ forms an outer layer encasing the core or wherein the material M₁ forms a core of the wire and the material M₂ forms an outer layer encasing the core.

4. The electrode for an electrosurgical handpiece as claimed in claim 1, wherein the wire is composed of a plurality of layers of the materials M₁ and M₂ in the manner of a skin, with the materials M₁ and M₂ enveloping each other, and the core of the wire being formed of the material M₁ or M₂.

5. The electrode for an electrosurgical handpiece as claimed in claim 1, wherein the core-layer structure of the wire consisting of the at least two materials M₁ and M₂ extends over the entire length of the wire.

6. The electrode for an electrosurgical handpiece as claimed in claim 1, wherein the wire is composed of at least two pieces, namely a first end piece E₁ and at least one middle piece MST, with the middle piece MST being arranged against the end piece E₁, and the end piece E₁ being able to be coupled to the electrode support of the handpiece.

7. The electrode for an electrosurgical handpiece as claimed in claim 1, wherein the wire is composed of at least three pieces, namely a first end piece E₁ and a second end piece E₂ and at least one middle piece MST, with the middle piece MST being arranged between the two end pieces E₁ and E₂, and the end pieces E₁ and E₂ being able to be coupled to the electrode support of the handpiece.

8. The electrode for an electrosurgical handpiece as claimed in claim 6, wherein the middle piece MST of the wire has a core made of the material M₂ and an encasing outer layer made of the material M₁ or wherein the material M₁ forms the core of the middle piece MST and the material M₂ forms the encasing outer layer.

9. The electrode for an electrosurgical handpiece as claimed in claim 6, wherein the middle piece MST of the wire is composed of a plurality of layers of the materials M₁ and M₂ in the manner of a skin, with the materials M₁ and M₂ enveloping each other, and the core of the wire being formed of the material M₁ or M₂.

10. The electrode for an electrosurgical handpiece as claimed in claim 6, wherein the at least one end piece E₁, or the end pieces E₁ and E₂, is/are formed of an electrically conductive or an electrically non-conductive material M₃.

11. The electrode for an electrosurgical handpiece as claimed in claim 6, wherein one of the ends of each of the end pieces E₁ and E₂ is electrically conductively and mechanically connected to the middle piece MST.

12. The electrode for an electrosurgical handpiece as claimed in claim 6, wherein one of the ends of one of the end pieces E₁ or E₂ in each case is connected to the middle piece MST in an electrically insulated and mechanical manner.

13. The electrode for an electrosurgical handpiece as claimed in claim 11, wherein the end pieces E₁ and/or E₂ are glued to, welded to, clamped to or plugged together with the middle piece MST, or are crimped or welded to the latter by means of a sleeve.

14. The electrode for an electrosurgical handpiece as claimed in claim 11, wherein the middle piece MST is formed of the material M₁ or M₂ and the sleeve is formed complementary thereto of the material M₂ or M₁.

15. The electrode for an electrosurgical handpiece as claimed in claim 1, wherein the wire has a diameter of 0.2 mm to 1 mm.

16. The electrode for an electrosurgical handpiece as claimed in claim 1, wherein the outer layer encasing the core, or the sleeve, has a layer thickness of 3 μm to 0.2 mm.

17. The electrode for an electrosurgical handpiece as claimed in claim 1, wherein the material M₁ is platinum, a platinum alloy, platinum-iridium or platinum-tungsten, and/or the material M₂ is tungsten, tantalum or molybdenum, and/or the material M₃ is steel, stainless steel or copper or a copper alloy or an electrical insulator.

18. An electrosurgical handpiece, having an electrode as claimed in claim 1.
